(19) United States
(12) Patent Application Publication
(10) Pub. No.: US 2024/0398688 A1
(43) Pub. Date: Dec. 5, 2024

(54) ANHYDROUS COSMETIC COMPOSITION

(71) Applicant: L’ORÉAL, Paris (FR)

(72) Inventors: Christopher PANG, Nanuet, NY (US); Hubert Tunchiao Lam, Berkeley Heights, NJ (US); Yan L. Lee, Elmwood Park, NJ (US)

(73) Assignee: L’ORÉAL, Paris (FR)

(21) Appl. No.: 18/203,798

(22) Filed: May 31, 2023

(51) Int. Cl. A61K 8/88; A61K 8/73; A61Q 1/06

(52) U.S. Cl. CPC A61K 8/88; A61K 8/731; A61Q 1/06; A61K 2800/31; A61K 2800/412; A61K 2800/42; A61K 2800/48; A61K 2800/592; A61K 2800/612

(57) ABSTRACT

Anhydrous cosmetic compositions that provide improved dispersion of pigments and fillers, and solids spreading, as well as providing a matte color or mattifying effect, where the product may have a stable hardness. The composition may include a colorant, one or more oils, a synthetic polyamide, cellulose particles, and may be free of a silicone elastomer. The cellulose particles may be untreated cellulose particles having a particle size of 3.5 μm or less, cellulose particles treated with a salt and having a particle size of 10 μm or less, or a combination thereof.

### FIELD OF THE INVENTION

The present invention relates to cosmetic compositions, and specifically to cosmetic lip compositions utilizing synthetic polyamide (e.g., nylon) and cellulose to provide improved feel, dispersion, spreading, and a hardness that is stable over time.

### BACKGROUND

There is a demand for cosmetic products that are environmentally sustainable. Such products are preferably free of microplastics. Microplastics often provide useful properties to a cosmetic formulation that can be challenging to replace. For example, certain microplastics are added to provide a desired hardness to the formulation that is stable over time, and that also impacts the feel, dispersion, and/or spreading of pigments. Replacing such a material requires a complete reformulation, and often results in compromising either the feel, dispersion, spreading, and/or the hardness.

Therefore, it is desirable to have a formula that replaces a microplastic thickening agent with a sustainable material, that can maintain or improve feed, dispersion, and spreading, while also providing a hardness that is stable over time.

### BRIEF SUMMARY

In various aspects, an anhydrous cosmetic composition may be provided. The composition may include a synthetic polyamide such as nylon. The composition may include cellulose particles. The composition may include a colorant and one or more oils. The composition may be free of a silicone elastomer. The cellulose particles may be untreated cellulose particles having a particle size of 3.5 μm or less, cellulose particles treated with a salt or an ester and having a particle size of 20 μm or less, or a combination thereof.

The cellulose particles may be present in a total amount of, e.g., no more than 5% by weight of the anhydrous cosmetic composition. A ratio of the total amount of synthetic polyamide by weight to the total amount of the cellulose particles may be 1:4-1:1.5.

The salt may be a metal salt of a fatty acid, such as, e.g., magnesium stearate. The ester may be a hydrogenated seed oil, such as, e.g., jojoba esters.

The one or more oils may include a hydrocarbon-based oil. The one or more oils may include a silicone oil. The silicone oil(s) may be present in a total amount, by weight, that is greater than the total amount of hydrocarbon-based oil(s). The total amount of hydrocarbon-based oil and silicone oil, combined, may be at least 40% by weight of the anhydrous cosmetic composition;

The composition may include a wax. The total amount of wax(es) may be at least 10% by weight of the anhydrous cosmetic composition. The composition may include a fatty ester. The total amount of fatty ester(s) may be at least 20% by weight of the anhydrous cosmetic composition The composition may include a filler. The filler may be present in a total amount of at least 5% by weight of the anhydrous cosmetic composition.

In some embodiments, the anhydrous cosmetic composition may consist of the synthetic polyamide, the cellulose particles, the colorant, the hydrocarbon-based oil, the silicone oil, the wax, the fatty ester, the filler, and optionally less than 1% by weight of all other materials.

In various aspects, a method for providing matte color delivery with a lipstick having stable hardness may be provided. The method may include providing an anhydrous cosmetic lipstick composition base that includes one or more colorants. The composition base may be free of a silsesquioxane elastomer. The method may include adding a synthetic polyamide, as well as cellulose particles, to the anhydrous cosmetic lipstick composition base. The cellulose particles may be untreated cellulose particles having a particle size of 3.5 μm or less, cellulose particles treated with a salt or an ester and having a particle size of 20 μm or less, or a combination thereof.

### DETAILED DESCRIPTION

As used herein, articles such as “a” and “an” when used in a claim, are understood to mean one or more of what is claimed or described.

As used herein, the expression “at least one” means one or more and thus includes individual components as well as mixtures/combinations.

All percentages listed are by weight unless otherwise noted.

As used herein, the term “about [a number]” is intended to include values rounded to the appropriate significant digit. Thus, “about 1” would be intended to include values between 0.5and 1.5, whereas “about 1.0” would be intended to include values between 0.95 and 1.05.

As used herein, the term “between” two number is intended to be inclusive of the bounding numbers. Thus, between 0 and 1 would include both 0 and 1.

As used herein, the terms “include”, “includes” and “including” are meant to be non-limiting.

As used herein, the term “substantially free [of an ingredient]” means that the composition contains less than 1% of the identified ingredient. The term “free [of an ingredient]” means the composition contains less than 0.1% of the identified ingredient, preferably below detectable amounts of the identified ingredient, and most preferably none of the identified ingredient.

For purposes of the compositions and methods of the present invention where the invention “consists essentially of” the identified ingredients and/or process steps, the “basic and novel properties” of such compositions and/or methods are “improved dispersion of pigments and/or fillers”, “improved solid spreading”, and “providing a matte or mattifying finish”, while “maintaining a stable hardness”.

In various aspects, an anhydrous cosmetic composition may be provided. The composition may include a synthetic polyamide. The composition may include cellulose particles. The composition may include a colorant. The composition may include one or more oils. The composition may include a wax. The composition may include a fatty ester. The composition may include a filler. Each of these will be discussed separately below. Additionally, the composition may be free, or substantially free, of a silicone elastomer, such as a silsesquioxane elastomer, such as a vinyl dimethicone/methicone silsesquioxane crosspolymer.

### Synthetic Polyamide

The composition may include one or more synthetic polyamides. In some embodiments, the composition may include a single synthetic polyamide. Various synthetic polyamide may be utilized, such as nylon-12. The nylon may have a melting point below 200° C. The synthetic polyamide may have a density of 1.005-1.05.

The synthetic polyamide may be present in a total amount of at least 0.5% by weight of the composition. The synthetic polyamide may be present in a total amount of at least 1% by weight of the composition. The synthetic polyamide may be present in a total amount of at least 1.5% by weight of the composition. The synthetic polyamide may be present in a total amount of no more than 5% by weight of the composition. The synthetic polyamide may be present in a total amount of no more than 4% by weight of the composition. The synthetic polyamide may be present in a total amount of no more than 3% by weight of the composition. The synthetic polyamide may be present in a total amount of no more than 2.5% by weight of the composition.

### Cellulose Particles.

The composition may include a plurality of cellulose particles. The composition may include one or more types of cellulose particles. In some embodiments, the composition may include a single type of cellulose particle. The cellulose particles may be (i) untreated cellulose particles having a particle size of 3.5 μm or less, (ii) cellulose particles treated with a salt or an ester and having a particle size of 20 μm or less, or (iii) a combination thereof.

The cellulose particles may be present in a total amount of at least 1% by weight of the composition. The cellulose particles may be present in a total amount of at least 2% by weight of the composition. The cellulose particles may be present in a total amount of at least 3% by weight of the composition. The cellulose particles may be present in a total amount of no more than 10% by weight of the composition. The cellulose particles may be present in a total amount of no more than 8% by weight of the composition. The cellulose particles may be present in a total amount of no more than 6% by weight of the composition. The cellulose particles may be present in a total amount of no more than 5% by weight of the anhydrous cosmetic composition.

To aid in providing the desired characteristics, it may be beneficial to maintain a ration of the total amount of synthetic polyamide (by weight) to the total amount of the cellulose particles (by weight). There may be more cellulose particles (by weight) than synthetic polyamide (by weight). For example, a ratio of the total amount of synthetic polyamide by weight to the total amount of the cellulose particles may be 1:4-1:1.5. In some embodiments, the ratio may be 1:3-1:1.5.

The cellulose particle sizes are preferably relatively small. As used herein, the “particle size” is understood to be the D50 volume average size, which corresponds to the particle size defined such that 50% by volume of the particles have a size less than D50.

The particle size may preferably be no more than 3.5 μm. For example, the number-average particle size may be no more than 3 μm. For example, the particle size may be no more than 2.5 μm. For example, the particle size may be no more than 2 μm. For example, the particle size may be no more than 1.5 μm. For example, the particle size may be no more than 1 μm.

The particle size appears to play a role in determining how stable the hardness of the composition is. Typically, when the particle sizes are too large, the hardness becomes unstable, and increases over time (e.g., from the date of manufacture to a date 6 months later). Harder products result in undesirable use characteristics. It was determined that by treating the cellulose with a particular treatment, slightly larger particle sizes can be utilized without the hardness becoming unstable. Specifically, by treating with a metal salt of a fatty acid, or an ester, the particle size that still maintains a stable hardness can be increased. In a preferred embodiment, the particles are treated with a metal salt.

In some embodiments, a value representative of a hardness (such as a CISA value, described below) of the anhydrous cosmetic composition does not change by more than 5% for six months, such as six months after manufacturing, when stored in a 25° C., 40% relative humidity (RH) stability chamber. As increase in hardness may be more acceptable than decreases in hardness, in some embodiments, the value representative of the hardness may not decrease by more than 5% or increase by more than 12%, for six months, such as six months after manufacturing, when stored in a 25 æC, 40% RH stability chamber.

The treatment is typically accomplished via traditional techniques, such as depositing the compound on a surface via spray coating techniques. This may also include, e.g., washing, filtering, drying, and pulverizing, etc. The treatment preferably results in a uniform, round coating covering the surface of the cellulose. In some embodiments, a thickness of the coating may be less than a radius of the cellulose particle. For example, if the cellulose particle has a radius of 4 μm (8 μm diameter), the coating may be, e.g., 1 μm thick, resulting in a particle with a total diameter of 10 μm.

Thus, for such treated cellulose particles, the particle size may be relatively larger than for untreated cellulose.

The particle size of treated cellulose particles may preferably be no more than 20 μm. In some embodiments, the particle size of treated cellulose particles may preferably be no more than 15 μm. In some embodiments, the particle size of treated cellulose particles may preferably be no more than 12 μm. In some embodiments, the particle size of treated cellulose particles may preferably be no more than 10 μm. In some embodiments, the particle size of treated cellulose particles may preferably be no more than 8 μm. In some embodiments, the particle size of treated cellulose particles may preferably be no more than 5 μm. In some embodiments, the particle size of treated cellulose particles may preferably be at least 1 μm. In some embodiments, the particle size of treated cellulose particles may preferably be at least 3 μm. In some embodiments, the particle size of treated cellulose particles may preferably be at least 5 μm. In some embodiments, the particle size of treated cellulose particles may preferably be at least 10 μm. In some embodiments, the particle size of treated cellulose particles may preferably be at least 12 μm. The particle size of treated cellulose particles may be between 1 μm and 20 μm. The particle size of treated cellulose particles may be between 1 μm and 15 μm. In some embodiments, the maximum particle size may depend on the treatment. For example, in some embodiments, a particle treated with a metal salt may have a maximum particle size of 10 μm, 11 μm, 12 μm, 13 μm, 14 μm, 15 μm, or 20 μm, while a particle treated with an ester may have a maximum particle size of 5 μm, 6 μm, 7 μm, 8 μm, 9 μm, or 10 μm.

Such salts may be an alkali earth metal salt. Each salt may include, e.g., magnesium or calcium. Each salt may be, e.g., magnesium stearate or magnesium myristate.

The salts may be present in a total of not more than 20% by weight of the total amount treated cellulose particles. The salts may be present in a total of not less than 5% by weight of the total amount treated cellulose particles. The salts may be present in a total of no more than 1% by weight of the anhydrous cosmetic composition. The salts may be present in a total of no more than 0.5% by weight of the anhydrous cosmetic composition.

The ester may be, e.g., an ester derived from a seed oil, such as a hydrogenated or transesterified seed oil, such as jojoba esters.

The cellulose particle may include only the cellulosic material and optionally the material(s) used to treatment the cellulose. The cellulosic material is preferably cellulose. In preferred embodiments, the cellulosic material may be free, or substantially free, of cellulose derivatives, such as methylcelluloses, hydroxyethylcelluloses, hydroxylpropylmethylcelluloses, carboxymethylcelluloses and their mixtures.

### Colorant

The colorant may include one or more colorants. The colorant may be a liposoluble colorant. The colorant may include dyes, pigments, nacres, lakes, goniochromatic coloring agents, or other materials known in the art for providing a color in a cosmetic.

Among the liposoluble colorants, particular mention can be made of Sudan Red, DC Red 17, DC Green 6, β-carotene, Sudan Brown, DC Yellow 11, DC Violet 2, DC orange 4 (CI: 15510, Na salt), DC Orange 5, Quinoline Yellow, Red 21, Red 27 and beta-carotene.

The term “pigments” should be understood to mean white or colored, mineral or organic particles, which are insoluble in an aqueous solution and are intended for coloring and/or opacifying the resulting film.

The pigments may be present in a proportion of 0.01% to 25% by weight, in particular from 0.01% to 20% by weight, with respect to the total weight of the cosmetic composition. The pigments can be chosen from mineral pigments, organic pigments, and composite pigments (i.e. pigments with a mineral and/or organic material base).

The pigments can be chosen from monochrome pigments, lacquers, nacres, pigments with an optical effect, such as reflective pigments and goniochromatic pigments.

Mineral pigments can be chosen from metal oxide pigments, chromium oxides, iron oxides, titanium oxide, zinc oxides, cerium oxides, zirconium oxides, manganese violet, Prussian blue, ultramarine blue, ferric blue, and mixtures thereof.

It can also be a pigment having a structure that may be, for example, of sericite/brown iron oxide/titanium dioxide/silica type.

The dye may also comprise a pigment having a structure that may, for example, be of the type of silica microbeads containing iron oxide.

The organic pigments can for example be:
cochineal carmine,
organic pigments with azoic, anthraquinonic, indigoid, xanthenic, pyrenic, quinolinic, triphenylmethane, fluorane colorants;
organic lacquers or insoluble salts of sodium, of potassium, of calcium, of barium, of aluminum, of zirconium, of strontium, of titanium, of acid colorants such as the azoic, anthraquinonic, indigoid, xanthenic, pyrenic, quinolinic, triphenylmethane, fluorane colorants. These colorants generally contain at least one carboxylic or sulfonic acid group;
melanic pigments.

Among the organic pigments, mention can be made of D&C Blue no. 4, D&C Brown no. 1, D&C Green no. 5, D&C Green no. 6, D&C Orange no. 4, D&C Orange no. 5, D&C Orange no. 10, D&C Orange no. 11, D&C Red no. 7 (Calcium salt of Lithol Rubine), D&C Red no. 17, D&C Red no. 21, D&C Red no. 22, D&C Red no. 27, D&C Red no. 28, D&C Red no. 30, D&C Red no. 31, D&C Red no. 33, D&C Red no. 34, D&C Red no. 36, D&C Violet no. 2, D&C Yellow no. 7, D&C Yellow no. 8, D&C Yellow no. 10, D&C Yellow no. 11, FD&C Blue no. 1, FD&C Green no. 3, FD&C Red no. 40, FD&C Yellow no. 5, FD&C Yellow no. 6.

Along the organic lacquers, mention can be made of organic lacquers supported by an organic support such as colophony or aluminum benzoate, for example. Preferably, among the organic lacquers, mention can in particular be made of those known under the following names: D&C Red no. 2 Aluminum lake, D&C Red no. 3 Aluminum lake, D&C Red no. 4 Aluminum lake, D&C Red no. 6 Aluminum lake, D&C Red no. 6 Barium lake, D&C Red no. 6 Barium/Strontium lake, D&C Red no. 6 Strontium lake, D&C Red no. 6 Potassium lake, D&C Red no. 7 Aluminum lake, D&C Red no. 7 Barium lake, D&C Red no. 7 Calcium lake, D&C Red no. 7 Calcium/Strontium lake, D&C Red no. 7 Zirconium lake, D&C Red no. 8 Sodium lake, D&C Red no. 9 Aluminum lake, D&C Red no. 9 Barium lake, D&C Red no. 9 Barium/Strontium lake, D&C Red no. 9 Zirconium lake, D&C Red no. 10 Sodium lake, D&C Red no. 19 Aluminum lake, D&C Red no. 19 Barium lake, D&C Red no. 19 Zirconium lake, D&C Red no. 21 Aluminum lake, D&C Red no. 21 Zirconium lake, D&C Red no. 22 Aluminum lake, D&C Red no. 27 Aluminum lake, D&C Red no. 27 Aluminum/Titanium/Zirconium lake, D&C Red no. 27 Barium lake, D&C Red no. 27 Calcium lake, D&C Red no. 27 Zirconium lake, D&C Red no. 28 Aluminum lake, D&C Red no. 30 lake, D&C Red no. 31 Calcium lake, D&C Red no. 33 Aluminum lake, D&C Red no. 34 Calcium lake, D&C Red no. 36 lake, D&C Red no. 40 Aluminum lake, D&C Blue no. 1 Aluminum lake, D&C Green no. 3 Aluminum lake, D&C Orange no. 4 Aluminum lake, D&C Orange no. 5 Aluminum lake, D&C Orange no. 5 Zirconium lake, D&C Orange no. 10 Aluminum lake, D&C Orange no. 17 Barium lake, D&C Yellow no. 5 Aluminum lake, D&C Yellow no. 5 Zirconium lake, D&C Yellow no. 6 Aluminum lake, D&C Yellow no. 7 Zirconium lake, D&C Yellow no. 10 Aluminum lake, FD&C Blue no. 1 Aluminum lake, FD&C Red no. 4 Aluminum lake, FD&C Red no. 40 Aluminum lake, FD&C Yellow no. 5 Aluminum lake and FD&C Yellow no. 6 Aluminum lake.

The pigments can be treated by a hydrophobic agent.

The hydrophobic treatment agent can be chosen from silicones such as methicones, dimethicones, perfluoroalkylsilanes; fatty acids such as stearic acid; metallic soaps such as aluminum dimyristate, the aluminum salt of hydrogenated tallow glutamate, perfluoroalkyl phosphates, perfluoroalkyl silanes, perfluoroalkyl silazanes, hexafluoropropylene polyoxides, polyorganosiloxanes comprising perfluoroalkyl perfluoropolyethers groups, amino acids; N-acylated amino acids or salts thereof; lecithin, isopropyl triisostearyl titanate and mixtures thereof.

The N-acylated amino acids may comprise an acyl group having 8 to 22 carbon atoms, such as for example a 2-ethyl hexanoyl, caproyl, lauroyl, myristoyl, palmitoyl, stearoyl, cocoyl group. The salts of these compounds may be aluminum, magnesium, calcium, zirconium, zinc, sodium, potassium salts. The amino acid may be for example lysine, glutamic acid, alanine.

The term “alkyl” mentioned in the above-mentioned compounds particularly denotes an alkyl group having 1 to 30 carbon atoms, preferably having 5 to 16 carbon atoms.

As used herein, the term “nacres” should be understood to mean iridescent or non-iridescent colored particles of any shape which are in particular produced by certain mollusks in their shell or else are synthesized and which exhibit a color effect by optical interference.

The nacres may be selected from pearlescent pigments such as titanium mica coated with iron oxide, titanium mica coated with bismuth oxychloride, titanium mica coated with chromium oxide, titanium mica coated with an organic dye, and pearlescent pigments based on bismuth oxychloride. This may also involve mica particles at the surface whereof are superposed at least two successive layers of metal oxides and/or of organic dyes.

By way of example of nacres, mention may also be made of natural mica coated with titanium oxide, with iron oxide, with natural pigment or with bismuth oxychloride.

Among the nacres available on the market, mention may be made of the TIMICA®, FLAMENCO®, and DUOCROME® nacres (based on mica) marketed by BASF, the TIMIRON® nacres marketed by Merck, and the nacres based on synthetic mica, SUNSHINE® marketed by SUN CHEMICAL.

The nacres may more particularly possess a yellow, pink, red, bronze, orange, brown, gold and/or copper color or glint.

By way of illustration of nacres which can be used in the context of the invention, mention may, in particular, be made of the gold nacres marketed, in particular, by BASF, under the name Brilliant gold 212G (TIMICA®), Gold 222C (CLOISSONE®), Sparkle gold (TIMICA®), Gold 4504 (CHROMALITE®) and Monarch gold 233X (CLOISSONE®); the bronze nacres, marketed, in particular, by MERCK under the name Bronze fine (17384) (COLORONA®) and Bronze (17353) (COLORONA®) and by BASF under the name Super bronze (CLOISSONE®); the orange nacres, in particular, under the name Orange 363C (CLOISSONE®) and Orange MCR 101 (COSMICA®) and by MERCK under the name Passion orange (COLORONA®) and Matte orange (17449) (MICRONA®); the brown-hued nacres marketed in particular by ENGELHARD under the name Nu-antique copper 340XB (Cloisonne) and Brown CL4509 (CHROMALITE®); the copper-glint nacres marketed in particular by ENGELHARD under the name Copper 340A (TIMICA®); the red-glint nacres marketed in particular by MERCK under the name Sienna fine (17386) (COLORONA®); the yellow-glint nacres marketed in particular by BASF under the name Yellow (4502) (CHROMALITE®); the gold-glint red-hued nacres marketed in particular by BASF under the name Sunstone G012 (GEMTONE®); the pink nacres under the name Tan opal G005 (GEMTONE®); the gold-glint black nacres under the name Nu-antique bronze 240 AB (TIMICA®), the blue nacres marketed in particular by MERCK under the name Matte blue (17433) (MICRONA®), the silver-glint white nacres marketed in particular by MERCK under the name XIRONA® Silver and the green-gold and pinkish orangish nacres marketed in particular by MERCK under the name Indian summer (XIRONA®) and mixtures thereof.

The cosmetic composition according to the invention may also contain at least one material with a specific optical effect.

This effect is different from a simple conventional hue effect, i.e. a unified and stabilized effect of the kind produced by conventional dyes, such as, for example, monochromatic pigments. For the purpose of the invention, the term “stabilized” signifies absence of an effect of variability of color with the angle of observation or in response to a temperature change.

For example, this material may be selected from particles having a metallic glint, goniochromatic coloring agents, diffracting pigments, thermochromatic agents, optical brighteners, and also fibers, in particular of the interference type. Of course, these various materials may be combined so as to provide the simultaneous manifestation of two effects, or even a new effect in accordance with the invention.

The metallic-glint particles that can be used in the invention are in particular chosen from:
particles of at least one metal and/or of at least one metal derivative,
particles comprising a single-substance or multi-substance, organic or mineral substrate, at least partially coated with at least one metallic-glint layer comprising at least one metal and/or at least one metal derivative, and
mixtures of said particles.

Among the metals that may be present in said particles, mention may, for example, be made of Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr and mixtures or alloys thereof (for example, bronzes and brasses) are preferred metals.

As used herein, the term “metal derivatives” denotes compounds derived from metals, in particular oxides, fluorides, chlorides and sulfides.

By way of illustration of these particles, mention may be made of aluminum particles, such as those marketed under the trade names STARBRITE 1200 EAC® pigments by Silberline, and METALURE® pigments by Eckart.

Mention may also be made of metal powders of copper or of alloy mixtures, metal pigments, such as aluminum or bronze, silica-coated aluminum particles, and metal alloy particles, such as silica-coated bronze (copper and zinc alloy).

The particles in question may also be particles comprising a glass substrate, such as those marketed under the trade name METASHINE® pigments by Nippon Sheet Glass.

The goniochromatic coloring agent may be selected, for example, from multilayer interference structures and liquid-crystal coloring agents.

Examples of symmetrical multilayer interference structures that may be used in compositions prepared according to the invention are, for example, the following structures: Al/SiO₂/Al/SiO₂/AI, pigments having this structure being marketed by Dupont; Cr/MgF₂/Al/MgF2/Cr, pigments having this structure being marketed under the trade name CHROMAFLAIR® by Flex; MoS2/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being marketed under the trade name SICOPEARL® by BASF; MoS2/SiO₂/mica-oxide/SiO₂/MoS2; Fe₂O₃/SiO₂/mica-oxide/SiO₂/Fe₂O₃; TiO₂/SiO₂/TiO₂ and TiO₂/Al2O3/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being marketed under the trade name XIRONA® by Merck. By way of example, these pigments may be the pigments with a silica/titanium oxide/tin oxide structure marketed under the name XIRONA® Magic by Merck, pigments with a silica/brown iron oxide structure marketed under the name XIRONA® Indian Summer by Merck and pigments with a silica/titanium oxide/mica/tin oxide structure marketed under the name XIRONA® Carribean Blue by Merck. Mention may also be made of the INFINITE COLORS® pigments from Shiseido. Depending on the thickness and the nature of the various layers, various effects are obtained. Thus, with the structure Fe₂O₃/SiO₂/Al/SiO₂/Fe₂O₃, the color changes from green-golden to red-gray for SiO₂ layers from 320 to 350 nm; from red to golden for SiO₂layers from 380 to 400 nm; from violet to green for SiO₂ layers from 410 to 420 nm; from copper to red for SiO₂layers from 430 to 440 nm.

By way of example of pigments with a polymeric multilayer structure, mention may be made of those marketed by 3M under the trade name COLOR GLITTER®.

Examples of liquid-crystal goniochromatic particles that may be used include those sold by Chenix, and also that marketed under the trade name HELICONE® HC by Wacker.

The colorant(s) may be present in a total amount of at least 1% by weight of the composition. The colorant(s) may be present in a total amount of at least 5% by weight of the composition. The colorant(s) may be present in a total amount of at least 10% by weight of the composition. The colorant(s) may be present in a total amount of no more than 20% by weight of the composition. The colorant(s) may be present in a total amount of no more than 18% by weight of the composition. The colorant(s) may be present in a total amount of no more than 15% by weight of the composition.

### Oils.

The composition may include one or more oils. The one or more oils may include hydrocarbon-based oil(s) and/or silicone oil(s). In some embodiments, the one or more oils may include both hydrocarbon-based oils and silicone oils.

As used herein, the term “oil” means a water-immiscible non-aqueous compound that is liquid at room temperature (25° C.) and at atmospheric pressure (760 mmHg).

### Hydrocarbon-Based Oil

The cosmetic composition may include a hydrocarbon-based oil. In some embodiments, the hydrocarbon-based oil may be present in a total amount no more than 15% by weight of the composition. In some embodiments, the hydrocarbon-based oil may be present in a total amount no more than 12% by weight of the composition. In some embodiments, the hydrocarbon-based oil may be present in a total amount no more than 10% by weight of the composition. In some embodiments, the hydrocarbon-based oil may be present in a total amount of at least 5% by weight of the composition. In some embodiments, the hydrocarbon-based oil may be present in a total amount of at least 6% by weight of the composition. In some embodiments, the hydrocarbon-In some embodiments, the hydrocarbon-based oil may be present in a total amount of at least 7% by weight of the composition. The hydrocarbon-based oil may be present in a total amount between 5% and 15% by weight. In some embodiments, the hydrocarbon-based oil may be present in a total amount between 7% and 12% by weight.

The term “oil” means any fatty substance which is in liquid form at room temperature (20-25° C.) and at atmospheric pressure (760 mmHg).

The term “hydrocarbon-based [material]” refers to the material mainly containing hydrogen and carbon atoms and possibly oxygen, nitrogen, sulfur and/or phosphorus atoms. In some embodiments, the hydrocarbon-based oil may be linear or branched. In some embodiments, the hydrocarbon-based oil is a hydrocarbon. Representative examples of hydrocarbon-based oils include oils containing from 8 to 16 carbon atoms, and especially branched C8-C16 alkanes (also known as isoparaffins), for instance isododecane (also known as 2,2,4,4,6-pentamethylheptane), and isohexadecane.

In some embodiments, the hydrocarbon-based oil may be apolar (therefore, formed only of carbon and hydrogen atoms).

The hydrocarbon-based oil may include linear or branched hydrocarbons of mineral or synthetic origin, such as petrolatum, polydecenes, hydrogenated polyisobutene such as Parleam®, squalane and liquid paraffins and mixtures thereof.

The hydrocarbon-based oil may be volatile or non-volatile.

The term “volatile” oil relates to an oil that is capable of evaporating on contact with the skin or a keratin fiber in less than one hour, at room temperature and atmospheric pressure. The volatile oil(s) are liquid at room temperature and have a non-zero vapor pressure, at room temperature and atmospheric pressure, ranging in particular from 0.13 Pa to 40 000 Pa (10⁻³ to 300 mmHg). The term “non-volatile” oil relates to oil which remains on the skin or the keratin fiber, at room temperature and atmospheric pressure, for at least several hours and which in particular has a vapor pressure of less than 10⁻³ mmHg (0.13 Pa).

In some embodiments, a plurality of hydrocarbon-based oils are present. The hydrocarbon-based oils may comprise or consist of at least one oil containing from 8 to 16 carbon atoms (such as isohexadecane), and at least one linear or branched hydrocarbons of mineral or synthetic origin (such as hydrogenated polyisobutene); in some embodiments, the hydrocarbon-based oils may be free, or substantially free, of other hydrocarbon-based oils. In some embodiments, the total amount of linear or branched hydrocarbons of mineral or synthetic origin (by weight) may be at least 3 times the total amount of oils containing 8-16 carbon atoms (by weight).

### Silicone Oil

The composition may include one or more silicone oils.

Non-limiting examples of silicone oils includes linear organopolysiloxanes such as dimethylpolysiloxane, methylphenylpolysiloxane, methylhydrogenopolysiloxane and the like; cyclic organopolysiloxanes such as octamethylcyclotetrasiloxane, decamethylcyclopentasiloxane, dodecamethylcyclohexasiloxane and the like; and mixtures thereof.

Preferably, the silicone oil is chosen from liquid polydialkylsiloxanes, in particular liquid polydimethylsiloxanes (PDMS) and liquid polyorganosiloxanes comprising at least one aryl group.

The polydialkylsiloxanes may be chosen from polydimethylsiloxanes comprising trimethylsilyl end groups, and polydimethylsiloxanes comprising dimethylsilanol end groups, known under the name dimethiconol (CTFA), and preferably polydimethylsiloxanes comprising trimethylsilyl end groups.

The polydialkylsiloxane chosen may be a mixture of dimethicone and dimethiconol (INCI name) available under the trade name Xiameter PMX-1503 FLUID® by the company Dow Corning.

These silicone oils may also be organomodified. The organomodified silicones that may be used according to the present invention are silicone oils as defined above comprising in their structure one or more organofunctional groups linked via a hydrocarbon-based group.

The organopolysiloxanes may be volatile or non-volatile.

Volatile or non-volatile silicone oils, such as volatile or non-volatile polydimethylsiloxanes (PDMS) containing a linear or cyclic silicone chain, which are liquid or pasty at ambient temperature, in particular cyclopolydimethylsiloxanes (cyclomethicones) such as cyclohexasiloxane; polydimethylsiloxanes containing oxyethylene, alkyl, alkoxy or phenyl groups that are pendent or at the end of the silicone chain, said groups containing from 2 to 24 carbon atoms; phenyl silicones such as phenyl trimethicones, phenyl dimethicones, phenyltrimethylsiloxydiphenylsiloxanes, diphenyl dimethicones, diphenylmethyldiphenyltrisiloxanes, 2-phenyl ethyl trimethylsiloxysilicates and polymethylphenylsiloxanes, may be used.

As examples of volatile silicone oils that may be used in the invention, mention may be made of:

volatile linear or cyclic silicone oils, in particular those with a viscosity 8 centistokes (8×10⁻⁶m²/s) and in particular containing from 2 to 7 silicon atoms, these silicones optionally comprising alkyl or alkoxy groups having from 1 to 10 carbon atoms. As volatile silicone oil that can be used in the invention, mention may particularly made of octamethylcyclotetrasiloxane, decamethylcyclopentasiloxane or cyclopentasiloxane, dodecamethylcyclohexasiloxane or cyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane.

The silicone oil may include dimethicone crosspolymer. Dimethicone crosspolymer may be present in an amount of no more than 5% by weight of the composition.

The silicone oil may include a plurality of types of silicone oils. For example, the silicone oil may comprise or consist of a polydialkylsiloxane, a phenyl silicone, and dimethicone crosspolymer.

In a preferred embodiment, a polydialkylsiloxane may be present in a total amount of at least 15% by weight of the composition. The polydialkylsiloxane may be present in a total amount of at least 20% by weight of the composition. The polydialkylsiloxane may be present in a total amount of no more than 30% by weight of the composition. The polydialkylsiloxane may be present in a total amount of no more than 25% by weight of the composition.

In a preferred embodiment, a phenyl silicone may be present in a total amount of at least 3% by weight of the composition. The phenyl silicone may be present in a total amount of at least 4% by weight of the composition. The phenyl silicone may be present in a total amount of at least 5% by weight of the composition. The phenyl silicone may be present in a total amount of no more than 10% by weight of the composition. The phenyl silicone may be present in a total amount of no more than 8% by weight of the composition. The phenyl silicone may be present in a total amount of no more than 6% by weight of the composition.

The silicone oil(s) may be present in a total amount, by weight, that is greater than the total amount of any hydrocarbon-based oil(s). In some embodiments, the silicone oil(s) may be present in a total amount, by weight, that is at least 3 times greater than the total amount of any hydrocarbon-based oil(s). In some embodiments, the silicone oil(s) may be present in a total amount, by weight, that is at least 3.5 times greater than the total amount of any hydrocarbon-based oil(s).

The total amount of hydrocarbon-based oil and silicone oil, combined, may be at least 30% by weight of the anhydrous cosmetic composition. The total amount of hydrocarbon-based oil and silicone oil, combined, may be at least 35% by weight of the anhydrous cosmetic composition. The total amount of hydrocarbon-based oil and silicone oil, combined, may be at least 40% by weight of the anhydrous cosmetic composition. The total amount of hydrocarbon-based oil and silicone oil, combined, may be no more than 60% by weight of the anhydrous cosmetic composition. The total amount of hydrocarbon-based oil and silicone oil, combined, may be no more than 55% by weight of the anhydrous cosmetic composition. The total amount of hydrocarbon-based oil and silicone oil, combined, may be no more than 50% by weight of the anhydrous cosmetic composition.

### Wax

The composition may include one or more waxes.

“Wax” as used herein means a lipophilic compound that is solid at room temperature (25¬∞ C.), with a solid/liquid reversible change of state, having a melting point of greater than or equal to 30¬∞ C., which may be up to 200¬∞ C., and in particular up to 120¬∞ C., including all ranges and subranges therebetween. Preferred waxes are those having a melting point of greater than or equal to 45¬∞ C. and in particular greater than or equal to 55¬∞ C.

Preferred waxes are chosen from waxes that are solid at room temperature of animal, plant, mineral and/or synthetic origin, and mixtures thereof. Preferably, the waxes are hydrocarbon-based or silicone waxes “Hydrocarbon-based wax” means a wax formed essentially from, or even constituted by, carbon and hydrogen atoms, and possibly oxygen and nitrogen atoms, and not containing any silicon or fluorine atoms. It may contain groups such as, for example, alcohol, ester, ether, carboxylic acid, amine and/or amide groups. “Silicone wax” means compounds comprising at least one silicon atom, preferably comprising Si-O groups.

In some embodiments, the wax comprises a polar wax.

In some embodiments, the polar wax is hydrocarbon-based. Suitable hydrocarbon-based waxes include, for example, beeswax, lanolin wax, rice bran wax, carnauba wax, candelilla wax, shellac wax; montan wax, orange wax and lemon wax, laurel wax, synthetic wax, and olive wax.

In some embodiments, the polar wax is an ester wax. “Ester wax” means a wax comprising at least one ester function or group. Examples of ester waxes include:
i) the waxes of formula RICOOR2 in which R1 and R2 represent linear, branched or cyclic aliphatic chains in which the number of atoms ranges from 10 to 50, which may contain a heteroatom such as O, N or P, and whose melting point ranges from about 25 to about 120¬∞ C., such as for example C20-C40 alkyl (hydroxystearyloxy)-stearate (the alkyl group containing from 20 to 40 carbon atoms), alone or as a mixture, or a C20-C40-alkyl stearate, Specific examples of such waxes are sold under the names Kester Wax K 82 P¬Æ, Hydroxypolyester K 82 P¬Æ, Kester Wax K 80 P¬Æ. Or Kester Wax K82H by the company Koster Keunen. Other specific examples include esters of polyethylene glycol and of montanic acid (octacosanoic acid), such as the wax Licowax KPS Flakes (INCI name: glycol montanate) sold by the company Clariant.
ii) bis(1,1, 1-trimethylolpropane) tetrastearate, sold under the name Hest 2T-4S¬Æ by the company Heterene,
iii) waxes of diesters of a dicarboxylic acid of general formula R3-(-OCO-R4-COO-R.5), in which R3 and R5 are identical or different, preferably identical, and represent a C4-C30 alkyl group (alkyl group comprising from 4 to 30 carbon atoms) and R4 represents a linear or branched C4-C.30 aliphatic group (alkyl group comprising from 4 to 30 carbon atoms), which may or may not contain one or more unsaturations, and is preferably linear and unsaturated,
iv) waxes obtained by hydrogenation of castor oil esterified with cetyl alcohol, sold under the names Phytowax ricin 16L64¬Æ and 22L73¬Æ by the company Sophim, and
v) hydrogenated oils such as hydrogenated jojoba oil.

In some embodiments, the polar wax is an oil-soluble polar modified polymer. “Polar modified polymer” as used herein refers to a hydrophobic homopolymer or copolymer which has been modified with hydrophilic unit(s). “Oil-soluble” as used herein means that the polar modified polymer is soluble in oil, and may be, e.g., a high carbon oil-soluble polar modified polymer, or a low carbon oil-soluble polar modified polymer.

“High carbon oil-soluble polar modified polymer” means a polymer containing more than 20 carbon atoms. Suitable monomers for the hydrophobic homopolymers and/or copolymers include, but are not limited to, cyclic, linear or branched, substituted or unsubstituted, C22-C40 compounds such as, C22-C28 compounds, C24-C26 compounds, C26-C28 compounds, and C30-C38 compounds, including all ranges and subranges therebetween. Preferably, the monomers are C24-26 compounds, C26-C28 compounds or C30-C38 compounds.

“Low carbon oil-soluble polar modified polymer” means a polymer containing 2 to 20 carbon atoms. Suitable monomers for the hydrophobic homopolymers and/or copolymers include, but are not limited to, cyclic, linear or branched, substituted or unsubstituted, C2-C20 compounds such as, for example, styrene, ethylene, propylene, isopropylene, butylene, isobutylene, pentene, isopentene, isoprene, hexene, isohexene, decene, isodecene, and octadecene, including all ranges and subranges therebetween. Preferably, the monomers are C2-C8 compounds, more preferably C2-C6 compounds, and most preferably C2-C4 compounds such as ethylene, propylene and butylene.

Suitable hydrophilic unit(s) for polar modified polymers include, but are not limited to, maleic anhydride, acrylates, alkyl acrylates such as, for example, methyl acrylate, ethyl acrylate, propyl acrylate, and butyl acrylate, and polyvinylpyrrolidone (PVP).

In some embodiments, the oil-soluble high carbon polar modified polymer waxes can be used, including, e.g., C26-C28 alpha olefin maleic acid anhydride copolymer waxes commercially available from Clariant under the trade name LICOCARE or LICOCENE. Specific examples of such waxes include products marketed by Clariant under the LicoCare name having designations such as CM 401, which is a maleic anhydride modified wax having a Mw of 2025 and a crystallinity of 11%, C30-C38 olefin/isopropylmaleate/maleic anhydride copolymer sold by Baker Hughes under the name Performa¬Æ V 1608, and C24-C26 alpha olefin acrylate copolymer wax commercially available from Clariant under the trade name LICOCARE CA301 LP3346 based on a polar backbone with C24-26 side chains with alternating ester and carboxylic acid groups.

In some embodiments, C2-C3 polar modified waxes can be used, including, e.g., polypropylene and/or polyethylene-maleic anhydride modified waxes (“PEMA,” “PPMA.” “PEPPMA”) commercially available from Clariant under the trade name LICOCARE or LICOCENE, Specific examples of such waxes include products marketed by Clariant under the LicoCare name having designations such as PP207. Other suitable polar modified polymers include, but are not limited to A-C 573 A (ETHYLENE-MALEIC ANHYDRIDE COPOLYMER; Drop Point, Mettler: 106¬∞ C.) from Honeywell, A-C 596 A (PROPYLENE-MALEIC ANHYDRIDE COPOLYMER; Drop Point, Mettler: 143¬≈ C.) from Honeywell, A-C 597 (PROPYLENE-MALEIC ANHYDRIDE COPOLYMER; Drop Point, Mettler: 141¬≈ C.) from Honeywell, ZeMac¬Æ copolymers (from VERTELLUS) which are 1:1 copolymers of ethylene and maleic anhydride, polyisobutylene-maleic anhydride sold under the trade name ISOBAM (from Kuraray), polyisoprene-graft-maleic anhydride sold by Sigma Aldrich, poly (maleic anhydride-octadecene) sold by Chevron Philips Chemical Co., poly (ethylene-co-butyl acrylate-co-maleic anhydride) sold under the trade name of Lotader (e.g. 2210, 3210, 4210, and 3410 grades) by Arkema, copolymers in which the butyl acrylate is replaced by other alkyl acrylates (including methyl acrylate [grades 3430, 4404, and 4503] and ethyl acrylate [grades 6200, 8200, 3300, TX 8030, 7500, 5500, 4700, and 4720) also sold by Arkema under the Lotader name, and isobutylene maleic anhydride copolymer sold under the name ACO-5013 by ISP.

In some embodiments, the wax is an alcohol wax. “Alcohol wax” means a wax comprising at least one alcohol function, i.e. comprising at least one free hydroxyl (OH) group. When alcohol waxes are used, preferably, the at least one long-chain alcohol wax has an average carbon chain length of between about 20 and about 60 carbon atoms, most preferably between about 30 and about 50 carbon atoms. Suitable examples of long-chain alcohol waxes include but are not limited to alcohol waxes commercially available from Baker Hughes under the Performacol trade name such as, for example, Performacol 350 (INCI Name: C20-C40 alcohols), 425 (INCI Name: C20-C40 alcohols) and 550 (INCI Name: C30-050 alcohols). Most preferably, the long-chain alcohol wax has a melting temperature range from about 93¬≈ C. to about 105¬≈ C. Another example of a commercial product comprising a suitable alcohol wax is Performacol 550-L Alcohol from New Phase Technologies.

In some embodiments, the wax is an apolar wax. Apolar waxes are, in particular, hydrocarbon-based waxes formed solely from carbon and hydrogen atoms, and free of heteroatoms such as N, O and P. In particular, the term “apolar wax” means a wax that is formed exclusively from apolar wax and not from a mixture also comprising other types of waxes that are not apolar waxes.

Examples of apolar waxes include hydrocarbon-based waxes, for instance microcrystalline waxes, paraffin waxes, ozokerite and polyethylene waxes. Polyethylene waxes that may be mentioned include Performalene 500-L Polyethylene and Performalene 400 Polyethylene sold by New Phase Technologies. An ozokerite that may be mentioned is Ozokerite Wax SP 1020 P. Examples of microcrystalline waxes include Multiwax W 445¬Æ sold by the company Sonneborn and Microwax HW¬Æ and Base Wax 30540¬Æ sold by the company Paramelt. Examples of microwaxes that may be used in the compositions according to the invention as apolar waxes include polyethylene microwaxes such as those sold under the names Micropoly 200¬Æ, 220¬Æ, 220L¬Æ, and 250S¬Æ by the company Micro Powders.

If present, waxes may be present in the composition in an amount ranging from about 0.1% to about 50%, preferably from about 1% to about 40%, and preferably from about 5% to about 30% by weight of the total weight of the composition, including all ranges and subranges therebetween.

The cosmetic composition may include two or more waxes. The cosmetic composition may include three or more waxes. The cosmetic composition may include four or more waxes. The cosmetic composition may include five or more waxes. The cosmetic composition may contain at least one polar wax and at least one apolar wax. The cosmetic composition may include two or more apolar waxes. The cosmetic composition may include three or more apolar waxes. The cosmetic composition may include four or more apolar waxes.

The cosmetic composition may contain wax(es) in a total amount of at least 5% by weight of the composition. The cosmetic composition may contain wax(es) in a total amount of at least 7.5% by weight of the composition. The cosmetic composition may contain wax(es) in a total amount of at least 10% by weight of the composition. The cosmetic composition may contain wax(es) in a total amount of no more than 25% by weight of the composition. The cosmetic composition may contain wax(es) in a total amount of no more than 20% by weight of the composition. The cosmetic composition may contain wax(es) in a total amount of no more than 15% by weight of the composition.

### Fatty Ester

The composition may include one or more fatty esters.

The fatty esters may include one or more liquid fatty esters. The term “liquid fatty ester” means an ester that that is liquid at room temperature and atmospheric pressure (25° C., 1 atm) and which comprises in its structure at least one hydrocarbon-based chain containing at least 6 carbon atoms. Preferably, it has a melting point of less than or equal to about 10° C.

The liquid fatty esters may be esters of monoalcohols or of polyols with monocarboxylic or polycarboxylic acids, at least one of the alcohols and/or acids comprising at least one hydrocarbon-based chain containing at least 6 carbon atoms.

The liquid fatty ester may be chosen from esters of a fatty acid (at least 6 carbon atoms) and of a monoalcohol, more particularly from esters of a fatty monoacid and of a monoalcohol. Preferably, at least one of the alcohols and/or acids is branched. Preferably, the alcohol and/or the acid are saturated, and preferentially both are saturated. Preferentially, the liquid fatty ester is not oxyalkylenated.

The liquid fatty esters according to the invention are preferably of formula R1-COO-R2, in which:
R1 denotes a linear or branched, saturated or unsaturated, optionally mono- or polyhydroxylated hydrocarbon-based radical, containing from 5 to 31 carbon atoms, preferably containing from 7 to 21 carbon atoms, and
R2 denotes a linear or branched, saturated or unsaturated, optionally mono- or polyhydroxylated hydrocarbon-based radical, containing from 1 to 20 carbon atoms.

Preferably, R1 denotes a linear or branched alkyl (saturated) radical containing 7 to 21 carbon atoms, especially from 8 to 17 carbon atoms, and more preferably from 8 to 15 carbon atoms.

Preferably, R2 denotes a linear alkyl (saturated) radical containing 1 to 4 carbon atoms or a branched alkyl (saturated) radical containing from 3 to 20 carbon atoms, especially from 3 to 16 carbon atoms. More preferably, R2 denotes a branched saturated alkyl radical containing from 3 to 12 carbon atoms.

Non-limiting examples of monoesters of monoacids and of monoalcohols include ethyl laurate, butyl laurate, hexyl laurate, isohexyl laurate, isopropyl laurate, isoamyl laurate, methyl myristate, ethyl myristate, butyl myristate, isobutyl myristate, isopropyl myristate, 2-octyldodecyl myristate, 2-ethylhexyl monococoate (or octyl monococoate), 2-ethylhexyl isononanoate, ethyl palmitate, isopropyl palmitate, isobutyl palmitate, 2-ethylhexyl palmitate (or octyl palmitate), butyl stearate, isopropyl stearate, isobutyl stearate, isocetyl stearate, isostearyl isostearate, isopropyl isostearate, 2-ethylhexyl stearate (or octyl stearate), 2-ethylhexyl hydroxystearate (or octyl hydroxystearate), decyl oleate, isononyl isononanoate, isodecyl neopentanoate, tridecyl neopentanoate, isocetyl neopentanoate, isostearylneopentanoate, octyldodecyl neopentanoate and isoarachidyl neopentanoate, and mixtures thereof.

In a preferred embodiment, the monoester is isostearyl isostearate hydrocarbon.

The fatty ester may include an aromatic ester such as tridecyl trimellitate.

In some embodiments, the fatty ester may include an ester of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, preferably such as bis-diglyceryl polyacyladipate-2 (INCI name) sold under the brand name Softisan 649 by the company Cremer Oleo.

In some embodiments, the fatty ester may include a liquid fatty ester, an aromatic ester, and an ester of a glycerol oligomer. In some embodiments, the total amount (by weight of the liquid fatty ester may be less than the total amount (by weight) of the aromatic ester, which may be less than the total amount (by weight) of the ester of a glycerol oligomer. In some embodiments, the total amount (by weight) of the ester of a glycerol oligomer may be more than the combined amount (by weight) of the liquid fatty ester and the aromatic ester.

The fatty ester(s) may be present in a total amount of at least 10% by weight of the anhydrous cosmetic composition. The fatty ester(s) may be present in a total amount of at least 15% by weight of the anhydrous cosmetic composition. The fatty ester(s) may be present in a total amount of at least 20% by weight of the anhydrous cosmetic composition. The fatty ester(s) may be present in a total amount of at least 30% by weight of the anhydrous cosmetic composition. The fatty ester(s) may be present in a total amount of at least 35% by weight of the anhydrous cosmetic composition. The fatty ester(s) may be present in a total amount of at least 40% by weight of the anhydrous cosmetic composition.

### Filler

The composition may include one or more fillers. The filler may be, e.g., of organic or mineral nature. The term “filler” should be understood as meaning colourless or white solid particles of any form, which are in an insoluble form dispersed in the medium of the composition. These particles, of mineral or organic nature, give body or rigidity to the composition and/or softness and uniformity to the makeup.

The fillers used in the compositions according to the invention may be of lamellar, globular, spherical or fibrous form or of any other form intermediate between these defined forms.

The fillers according to the invention may or may not be surface-coated, and in particular they may be surface-treated with silicones, amino acids, fluorinated derivatives or any other substance which promotes the dispersion and the compatibility of the filler in the composition.

Examples of mineral fillers that may be mentioned include talc, mica, silica, hollow silica microspheres, kaolin, calcium carbonate, magnesium carbonate, hydroxyapatite, boron nitride, glass or ceramic microcapsules, or composites of silica and of titanium dioxide, for instance the TSG series sold by Nippon Sheet Glass.

Examples of organic fillers that may be mentioned include polyethylene powders, polymethyl methacrylate powders, polytetrafluoroethylene (Teflon) powders, acrylic acid copolymer powders (Polytrap from the company Dow Corning), lauroyl lysine, hollow polymer microspheres such as those of polyvinylidene chloride/acrylonitrile, for instance Expancel (Nobel Industrie), hexamethylene diisocyanate/trimethylol hexyllactone copolymer powder (Plastic Powder from Toshiki), silicone resin microbeads (for example Tospearl from Toshiba), synthetic or natural micronized waxes, metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate, Polypore® L 200(Chemdal Corporation), polyurethane powders, in particular powders of crosslinked polyurethane comprising a copolymer, said copolymer comprising trimethylol hexyllactone.

The filler may be present in a total amount of at least 3% by weight of the anhydrous cosmetic composition. The filler may be present in a total amount of at least 5% by weight of the anhydrous cosmetic composition. The filler may be present in a total amount of no more than 10% by weight of the anhydrous cosmetic composition. The filler may be present in a total amount of no more than 8% by weight of the anhydrous cosmetic composition.

In some embodiments, the anhydrous cosmetic composition may consist of the synthetic polyamide, the cellulose particles, the colorant, the hydrocarbon-based oil, the silicone oil, the wax, the fatty ester, the filler, and optionally less than 1% by weight of all other materials. For example, the composition may include less than 1% total of fragrances, antioxidants, coatings of various pigments, etc.

In various aspects, a method for providing matte color delivery with a lipstick having stable hardness may be provided. The method may include providing an anhydrous cosmetic lipstick composition base that includes one or more colorants. The composition base may be free of a silicone elastomer. The method may include adding a synthetic polyamide, as well as cellulose particles, to the anhydrous cosmetic lipstick composition base. The cellulose particles may be untreated cellulose particles having a relatively smaller particle size, cellulose particles treated with a salt or an ester and having a relatively larger particle size, or a combination thereof.

### EXAMPLES

Several lipstick compositions were created, as described in Tables 1 and 2, below. Two comparative formulas (C1, C2) and three exemplary formulas (E1, E2, E3) are listed. To produce each formulation, a base formula is created by grinding the pigments with a mixture of the hydrocarbon-based oils, silicone oils, fatty esters, and fillers to create a pigment grind. The pigment grind and waxes are then placed in a kettle and heated up to 95° C. and mixed until homogenous. Once homogenous, remainder of the base formula materials are added and mixed until homogenous. Then, the modifiers (e.g., synthetic polyamide and cellulose) are added and mixed until homogenous. Once completed, the hot formulations are poured into lipstick molds and cooled to room temperature before transferring to a chilled environment and then placed in lipstick packs.

TABLE 1
(Comparative Formulas)

| Material | C1 | C2 |
|---|---|---|
| Base Formula | | |
| Treated Pigments | 10-20% | 10-20% |
| Hydrocarbon-Based Oils | 5-10% | 5-10% |
| Silicone Oils | 30-40% | 30-40% |
| Fatty Esters | 20-30% | 20-30% |
| Filler | 5-10% | 5-10% |
| Waxes | 5-15% | 5-15% |
| Additives | <1% | <1% |
| Modifiers | | |
| Nylon-12 | 1.5-2.5% | 1.5-2.5% |
| Silsesquioxane Resin | 3-5% | — |
| Untreated Cellulose (10 μm) | | 3-5% |
| Untreated Cellulose (3.5 μm) | — | — |
| Treated Cellulose (Magnesium | — | — |
| Stearate, 12-15 μm) | | |
| Treated Cellulose | — | — |
| (Jojoba Esters, 12-15 μm) | | |

TABLE 2
(Exemplary Formulas)

| Material | E1 | E2 | E3 |
|---|---|---|---|
| Base Formula | | | |
| Treated Pigments | 10-20% | 10-20% | 10-20% |
| Hydrocarbon-Based Oils | 5-10% | 5-10% | 5-10% |
| Silicone Oils | 30-40% | 30-40% | 30-40% |
| Fatty Esters | 20-30% | 20-30% | 20-30% |
| Filler | 5-10% | 5-10% | 5-10% |
| Waxes | 5-15% | 5-15% | 5-15% |
| Additives | <1% | <1% | <1% |
| Polyamides and Cellulose | | | |
| Nylon-12 | 1.5-2.5% | 1.5-2.5% | 1.5-2.5% |
| Silsesquioxane Resin | — | — | |
| Untreated Cellulose (10 μm) | — | — | |
| Untreated Cellulose (3.5 μm) | 3-5% | — | |
| Treated Cellulose | — | 3-5% | |
| (Magnesium Stearate, | | | |
| 12-15 μm) | | | |
| Treated Cellulose | — | — | 3-5% |
| (Jojoba Esters, 12-15 μm) | | | |

### Evaluations

The various lip products were evaluated for, inter alia, coverage and spreading, and ciasaillement (“CISA”) values over time. Those values can be seen in Table 3, below. Coverage and spreading were subjectively determined and compared by a panel of trained assessors. CISA values are determined by measuring the grams of force (i.e., maximum grams of force) needed for a wire moving at a set rate of speed to cut a product with a defined shape (such as a “bullet” shape) in half. Thus, lower CISA values represent “softer” products, and higher CISA values represent “harder” products.

TABLE 3

| Test | C1 | C2 | E1 | E2 | E3 |
|---|---|---|---|---|---|
| Coverage/Spreading | Ok | Ok | Ok | V. Good | Good |
| CISA (t = 0) | 198.5 | 159.1 | 166.8 | 185.9 | 166.1 |
| CISA | 172.5 | 174.5 | 167 | 175.1 | 174 |
| (t = 1 month) | | | | | |
| CISA | 176 | 197 | 171 | 185 | 185 |
| (t = 6 months) | | | | | |

As seen in the table above, exemplary formulas have similar to much improved coverage and spreading, as compared to a baseline product (here, C1). Further, such exemplary formulas also have a hardness (see CISA value) that is generally stable over time (e.g., the CISA value at 6 months that has not decreased by more than 5%, and has increased by less than 12% of the starting value, and preferably less than 5% of the starting value). Of particular note, replacing the silsesequoxane resin in the baseline product with larger untreated cellulose particles (C2) resulted in a product with an unstable hardness.

Further, replacing the resin in the baseline particles with larger cellulose particles treated with esters (E3) resulted in a less stable hardness than those treated with metal salts (E2). Thus, it is clear that to achieve a stable hardness, you may preferably utilize small uncoated particles (e.g., 3.5 μm or smaller), slightly larger particles treated with an ester (e.g., 5 μm, 8 μm, or 10 μm or smaller), and/or larger particles coated with a metal salt (e.g., 10 μm, 12 μm, 15 μm, or 20 μm or smaller).

Those skilled in the art will recognize or be able to ascertain using no more than routine experimentation many equivalents to the specific embodiments of the invention described herein. Such equivalents are intended to be encompassed by the following claims.

What is claimed is:

1. An anhydrous cosmetic composition, comprising:
a synthetic polyamide;
cellulose particles, comprising:
untreated cellulose particles having a particle size of 3.5 μm or less;
cellulose particles treated with a salt or an ester and having a particle size of 15 μm or less; or
a combination thereof; and
a colorant; and
one or more oils;
wherein the anhydrous cosmetic composition is free of a silsesquioxane elastomer.

2. The anhydrous cosmetic composition according to claim 1, wherein the salt is a metal salt of a fatty acid.

3. The anhydrous cosmetic composition according to claim 2, wherein the metal salt of a fatty acid is magnesium stearate.

4. The anhydrous cosmetic composition according to claim 1, wherein the one or more oils comprises a hydrocarbon-based oil.

5. The anhydrous cosmetic composition according to claim 4, wherein the one or more oils further comprises a silicone oil.

6. The anhydrous cosmetic composition according to claim 5, wherein the silicone oil is present in a total amount, by weight, that is greater than the total amount of the hydrocarbon-based oil.

7. The anhydrous cosmetic composition according to claim 5, further comprising a wax.

8. The anhydrous cosmetic composition according to claim 7, further comprising a fatty ester.

9. The anhydrous cosmetic composition according to claim 8, further comprising a filler.

10. The anhydrous cosmetic composition according to claim 9, wherein:
the cellulose particles are present in a total amount of no more than 5% by weight of the anhydrous cosmetic composition; and
a ratio of the total amount of synthetic polyamide by weight to the total amount of the cellulose particles is 1:4-1:1.5.

11. The anhydrous cosmetic composition according to claim 10, wherein:
the total amount of hydrocarbon-based oil and silicone oil, combined, is at least 40% by weight of the anhydrous cosmetic composition;
the total amount of fatty ester is at least 20% by weight of the anhydrous cosmetic composition; and
the total amount of wax is at least 10% by weight of the anhydrous cosmetic composition.

12. The anhydrous cosmetic composition according to claim 11, wherein the filler is present in a total amount of at least 5% by weight of the anhydrous cosmetic composition.

13. The anhydrous cosmetic composition according to claim 9, wherein the anhydrous cosmetic composition consists of:
the synthetic polyamide;
the cellulose particles;
the colorant;
the hydrocarbon-based oil;
the silicone oil;
the wax;
the fatty ester;
the filler; and
optionally less than 1% by weight of all other materials.

14. The anhydrous cosmetic composition according to claim 1, wherein a value representative of a hardness of the anhydrous cosmetic composition does not change by more than 5% for six months.

15. A method for providing matte color delivery with a lipstick having stable hardness, comprising:
providing an anhydrous cosmetic lipstick composition base comprising one or more colorants, the anhydrous cosmetic lipstick composition base being free of a silsesquioxane elastomer; and
adding a synthetic polyamide and cellulose particles to the anhydrous cosmetic lipstick composition base, where the cellulose particles are:
untreated cellulose particles having a particle size of 3.5 μm or less;
cellulose particles treated with a salt or an ester and having a particle size of 15 μm or less; or
a combination thereof.
